(19) United States
(12) Patent Application Publication
(10) Pub. No.: US 2008/0249184 A1
(43) Pub. Date: Oct. 9, 2008

(54) Methods for modulating neuronal cell death

(75) Inventors: Francine Gervais, Ile Bizard (CA); Louis R. Lamontagne, Orleans (CA)

Correspondence Address: Lahive/ Neurochem, One Post Office Square, Boston, MA, 02109, US

(73) Assignee: Bellus Health (International) Limited, Lausanne (CH)

(21) Appl. No.: 12/157,284

(22) Filed: Jun. 9, 2008

Related U.S. Application Data
(63) Continuation of application No. 10/654,863, filed on Sep. 3, 2003.
Application No. 10/654,863 is a continuation of application No. 09/874,543, filed on Jun. 4, 2001, now abandoned.
Application No. 09/874,543 is a continuation-in-part of application No. 09/312,442, filed on May 14, 1999, now abandoned.
Application No. 09/312,442 is a continuation-in-part of application No. 09/248,396, filed on Feb. 10, 1999, now abandoned.
(60) Provisional application No. 60/085,571, filed on May 15, 1998.
(60) Provisional application No. 60/074,295, filed on Feb. 11, 1998.

(51) Int. Cl. A61K 31/185; A61K 31/145; A61P 25/28

(52) U.S. Cl. 514/665

(57) ABSTRACT

The invention provides methods of inhibiting Aβ-induced neuronal cell death. The invention further provides methods of providing neuroprotection to a subject and methods of treating a disease state characterized by Aβ-induced neuronal cell death in a subject. Methods of inhibiting p75 receptor mediated neuronal cell death, as well as methods of treating a disease state in a subject characterized by p75 receptor mediated neuronal cell death are provided.

### RELATED APPLICATIONS

This application is a continuation of copending application Ser. No. 10/654,863, filed on Sep. 3, 2003, which is a continuation of copending application Ser. No. 09/874,543, filed Jun. 4, 2001 which is a continuation-in-part of application Ser. No. 09/312,442, filed May 14, 1999, which claimed the benefit of priority U.S. Provisional Application No. 60/085,571, filed on May 15, 1998; which is also a continuation-in-part of application Ser. No. 09/248,396, filed Feb. 10, 1999, which claimed the benefit of priority of U.S. Provisional Application No. 60/074,295, filed on Feb. 11, 1998. The entire contents of all the aforementioned documents are incorporated herein by reference.

### FIELD OF THE INVENTION

This invention relates to methods for modulating neuronal cell death.

### BACKGROUND OF THE INVENTION

Amyloid-β (Aβ) is a neurotoxic peptide which is implicated in the pathogenesis of Alzheimer's Disease. In fact, extracellular deposition of Aβ peptide in specific regions of the brain is one of the hallmarks of Alzheimer's Disease. Aβ peptide is derived from a normal proteolytic cleavage of the precursor protein, the Amyloid-β precursor protein (β APP). Once deposited into the brain, the Aβ peptide forms senile plaques which have been found in greater numbers in the brains of patients with Alzheimer's Disease. The A β peptide has also been shown to infiltrate cerebrovascular walls and cause angiopathy. A progressive neuronal cell loss accompanies the deposition of Aβ amyloid fibrils in senile plaques. The Aβ peptide has been shown by several groups to be highly toxic to neurons. The amyloid plaques are directly associated with reactive gliosis, dystrophic neurites and apoptotic cells, suggesting that plaques induce neurodegenerative changes. In vitro, Aβ has been shown to be necrotic in rat PC-12 cells while it induces apoptosis in primary hippocampal culture from fetal rat and in the predifferentiated human neurotype SH-SY5Y cell line (Li et al. (1996) Brain Research 738:196-204).

Neurodegeneration associated with AD has been linked to the presence of fibrillary Aβ. Numerous reports have shown that Aβ fibrils can induce neurodegeneration. It has been hypothesized that such an activity was due to the acquisition of the β-sheet structure of Aβ. Non-fibrillar Aβ has also been shown to be cytotoxic to neurons. La Ferla et al. ((1997) J. Clin. Invest. 100(2):310-320) have recently shown that when neuronal cells are exposed in vitro to soluble Aβ they can become apoptotic. Once internalized, the Aβ peptide gets stabilized and induces DNA fragmentation, which is characteristic of apoptosis.

One major event in the formation of β-sheet fibrils is the binding of the Aβ peptide to the sulfated proteoglycans present at the cell surface. Basement membrane glycosaminoglycans (GAGs) have been shown to interact with all types of amyloidotic proteins. It has been suggested that the interaction of GAGs with an Aβ peptide induces conformational changes in favoring aggregation and formation of insoluble fibrils.

Nerve growth factor (NGF) has also been shown to potentiate the neurotoxicity of Aβ on differentiated hippocampal neurons in culture (Yankner B. A. et al. (1990) Proc. Natl. Acad. Sci. 87:9020-23). It has been suggested that β-amyloid deposits may cause induction of NGF receptor in neuronal cell types, typically unresponsive to NGF.

The mechanisms and specific molecules involved in neuronal cell death, e.g., Aβ peptide-induced neuronal cell death, still remain uncertain. As a result, to date, effective treatments for states associated with neuronal cell death, e.g., neurodegenerative disorders, have not been developed. Accordingly, methods for inhibiting neuronal cell death are still needed.

### SUMMARY OF THE INVENTION

The present invention provides methods for inhibiting neuronal cell death, e.g., Aβ-induced neuronal cell death and/or p75 receptor-mediated neuronal cell death. The present invention is based, at least in part, on the discovery that compounds which interfere with the association of the Aβ peptide, e.g., the association of the Aβ peptide to the sulfate GAGs present at the cell surface, and prevent the triggering of neuronal cell apoptosis or necrosis.

Accordingly, this invention pertains to a method of inhibiting Aβ-induced neuronal cell death. The method includes contacting a neuronal cell with an Aβ-interferer, such that neuronal cell death is inhibited. The Aβ-interferer can interfere with the ability of the Aβ peptide to form amyloid fibrils and/or with the ability of the Aβ peptide to bind to a cell surface molecule. The cell surface molecule can be, for example, a neurotrophic receptor, e.g., the apoptosis-related p75 receptor; a protein presented by plasma protein, e.g., RAGE; or a glycosaminoglycan. The Aβ peptide can be either in soluble form or in a fibril form.

In one embodiment, the Aβ-interferer is selected from the group consisting of ethanesulfonic acid, 1,2-ethanedisulfonic acid, 1-propanesulfonic acid, 1,3-propanedisulfonic acid, 1,4-butanedisulfonic acid, 1,5-pentanedisulfonic acid, 2-aminoethanesulfonic acid, 4-hydroxybutane-1-sulfonic acid, and pharmaceutically acceptable salts thereof. In other preferred embodiments, the Aβ-interferer is selected from the group consisting of 1-butanesulfonic acid, 1-decanesulfonic acid, 2-propanesulfonic acid, 3-pentanesulfonic acid, 4-heptanesulfonic acid, and pharmaceutically acceptable salts thereof. In yet further preferred embodiments, the Aβ-interferer is 1,7-dihydroxy-4-heptanesulfonic acid, 3-amino-1-propanesulfonic acid, or a pharmaceutically acceptable salt thereof. In an other embodiment the Aβ is a peptide or a peptidomimetic which interact with specific regions of the Aβ peptide such as the regions responsible for cellular adherence (aa 10-16), GAG binding site region (13-16) or the region responsible for the β-sheet formation (16-21). These peptides are the d-stereoisomers of the Aβ or complementary image of the Aβ peptide.

Another aspect of the invention pertains to a method of providing neuroprotection to a subject, comprising administering an Aβ-interferer to the subject, such that neuroprotection is provided.

In one embodiment, the Aβ-interferer interferes with the ability of the Aβ peptide to bind to a cell surface molecule, e.g., a neurotrophic receptor such as the apoptosis-related p75 receptor; a protein presented by plasma protein, e.g., RAGE; or a glycosaminoglycan. The Aβ peptide can be either in soluble form or in a fibril form.

In one embodiment, the Aβ-interferer is selected from the group consisting of ethanesulfonic acid, 1,2-ethanedisulfonic acid, 1-propanesulfonic acid, 1,3-propanedisulfonic acid, 1,4-butanedisulfonic acid, 1,5-pentanedisulfonic acid, 2-aminoethanesulfonic acid, 4-hydroxybutane-1-sulfonic acid, and pharmaceutically acceptable salts thereof. In other preferred embodiments, the Aβ-interferer is selected from the group consisting of 1-butanesulfonic acid, 1-decanesulfonic acid, 2-propanesulfonic acid, 3-pentanesulfonic acid, 4-heptanesulfonic acid, and pharmaceutically acceptable salts thereof. In yet further preferred embodiments, the Aβ-interferer is 1,7-dihydroxy-4-heptanesulfonic acid, 3-amino-1-propanesulfonic acid, or a pharmaceutically acceptable salt thereof.

In one embodiment, the Aβ-interferer is administered in a pharmaceutically acceptable formulation. The pharmaceutically acceptable formulation can be a dispersion system, for example a lipid-based formulation, a liposome formulation, or a multivesicular liposome formulation. The pharmaceutically acceptable formulation can also comprise a polymeric matrix, selected, for example, from synthetic polymers such as polyesters (PLA, PLGA), polyethylene glycol, poloxomers, polyanhydrides, and pluronics or selected from naturally derived polymers, such as albumin, alginate, cellulose derivatives, collagen, fibrin, gelatin, and polysaccharides. In other preferred embodiments, the pharmaceutically acceptable formulation provides sustained delivery of the Aβ-interferer to a subject.

Yet another aspect of the invention pertains to a method of treating a disease state characterized by Aβ-induced neuronal cell death in a subject. The method includes administering an Aβ-interferer to the subject, such that the disease state characterized by Aβ-induced neuronal cell death is treated.

Another aspect of the invention pertains to a method of inhibiting p75 receptor mediated neuronal cell death. The method includes contacting a neuronal cell with a therapeutic compound having the structure:

Q-[-Y⁻X³⁰]ₙ

wherein Y⁻ is an anionic group at physiological pH; Q is a carrier group; X⁺ is a cationic group; and n is an integer selected such that the biodistribution of the therapeutic compound for an intended target site is not prevented while maintaining activity of the therapeutic compound, provided that the therapeutic compound is not chondroitin sulfate A, such that neuronal cell death is inhibited.

A further aspect of the invention pertains to a method of providing neuroprotection to a subject. The method includes administering to the subject a therapeutic compound having the structure:

Q-[-Y⁻X⁺]ₙ

wherein Y⁻ is an anionic group at physiological pH; Q is a carrier group; X⁺ is a cationic group; and n is an integer selected such that the biodistribution of the therapeutic compound for an intended target site is not prevented while maintaining activity of the therapeutic compound, provided that the therapeutic compound is not chondroitin sulfate A, such that neuroprotection is provided.

In another aspect, the invention features a method of treating a disease state in a subject characterized by p75 receptor mediated neuronal cell death. The method includes administering to the subject a therapeutic compound having the structure:

Q-[-Y⁻X⁺]ₙ

wherein Y⁻ is an anionic group at physiological pH; Q is a carrier group; X⁺ is a cationic group; and n is an integer selected such that the biodistribution of the therapeutic compound for an intended target site is not prevented while maintaining activity of the therapeutic compound, provided that the therapeutic compound is not chondroitin sulfate A, such that the disease state characterized by p75 receptor mediated neuronal cell death is treated.

In yet another aspect, the invention features a method of inhibiting p75 receptor-mediated neuronal cell death. The method includes contacting a neuronal cell with a p75 receptor-interferer having the structure:

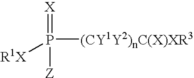

in which Z is XR² or R⁴; R¹ and R² are each independently hydrogen, a substituted or unsubstituted aliphatic group, an aryl group, a heterocyclic group, or a salt-forming cation; R³ is hydrogen, lower alkyl, aryl, or a salt-forming cation; R⁴ is hydrogen, lower alkyl, aryl or amino; X is, independently for each occurrence, O or S; Y¹ and y² are each independently hydrogen, halogen, alkyl, amino, hydroxy, alkoxy, or aryloxy; and n is an integer from 0 to 12, such that neuronal cell death is inhibited.

In a further aspect, the invention features a method of providing neuroprotection to a subject. The method includes administering to the subject a p75 receptor-interferer having the structure:

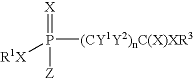

in which Z is XR² or R⁴; R¹ and R² are each independently hydrogen, a substituted or unsubstituted aliphatic group, an aryl group, a heterocyclic group, or a salt-forming cation; R³ is hydrogen, lower alkyl, aryl, or a salt-forming cation; R⁴ is hydrogen, lower alkyl aryl or amino; X is, independently for each occurrence, O or S; Y¹ and Y² are each independently hydrogen, halogen, alkyl, amino, hydroxy, alkoxy, or aryloxy; and n is an integer from 0 to 12, such that neuroprotection is provided.

In another aspect, the invention features a method of treating a disease state in a subject characterized by p75 receptor-mediated neuronal cell death. The method includes administering to the subject a p75 receptor-interferer having the structure:

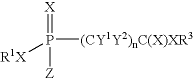

in which Z is XR² or R⁴; R¹ and R² are each independently hydrogen, a substituted or unsubstituted aliphatic group, an aryl group, a heterocyclic group, or a salt-forming cation; R³ is hydrogen, lower alkyl, aryl, or a salt-forming cation; R⁴ is hydrogen, lower alkyl, aryl or amino; X is, independently for each occurrence, O or S; Y¹ and Y² are each independently hydrogen, halogen, alkyl, amino, hydroxy, alkoxy, or aryloxy; and n is an integer from 0 to 12, such that said disease state characterized by p75 receptor mediated neuronal cell death is treated.

Other features and advantages of the invention will be apparent from the following detailed description, and from the claims.

### BRIEF DESCRIPTION OF THE DRAWINGS

FIG. 1 is a depiction of a bar graph showing the toxicity of Aβ(1-40) administered at a ratio of 1:1 with various Aβ-interferers, on PC-12 cells.

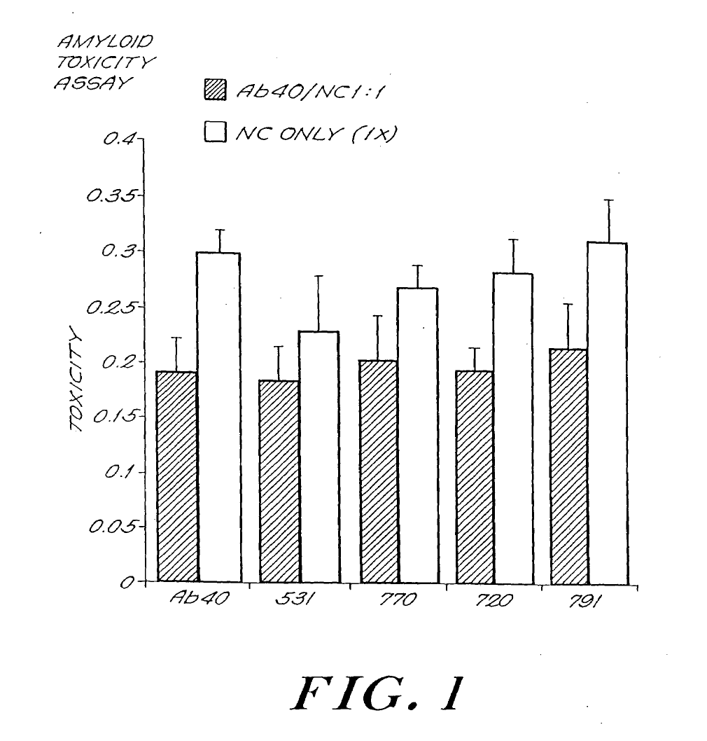

FIG. 2 is a depiction of a bar graph showing the toxicity of Aβ(1-40) administered at a ratio of 1:2 with various Aβ-interferers, on PC-12 cells.

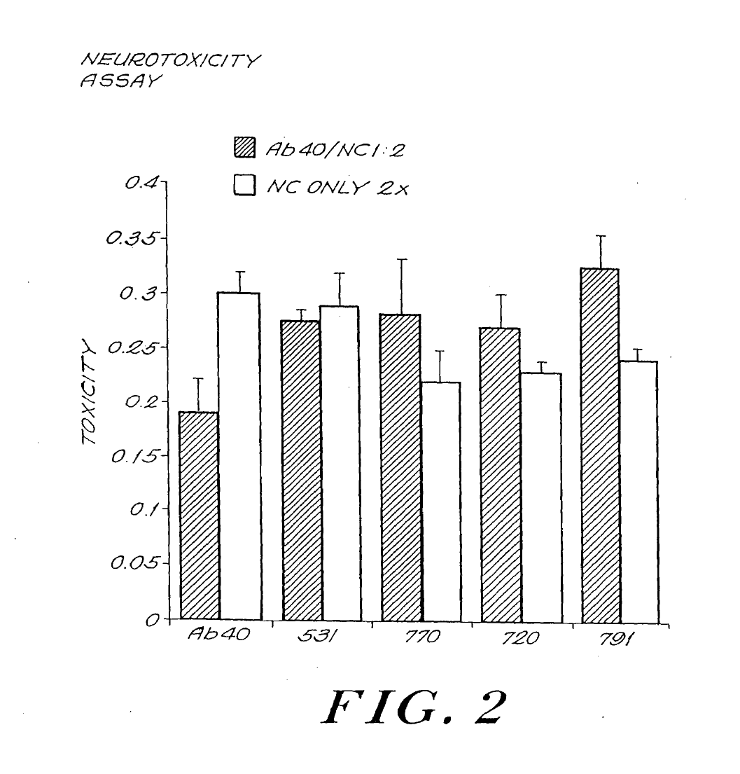

FIG. 3 is a depiction of a bar graph showing the % cell survival of differentiated PC-12 cells treated with Aβ(1-40) and various Aβ-interferers at a 1:2 and 1:1 ratio.

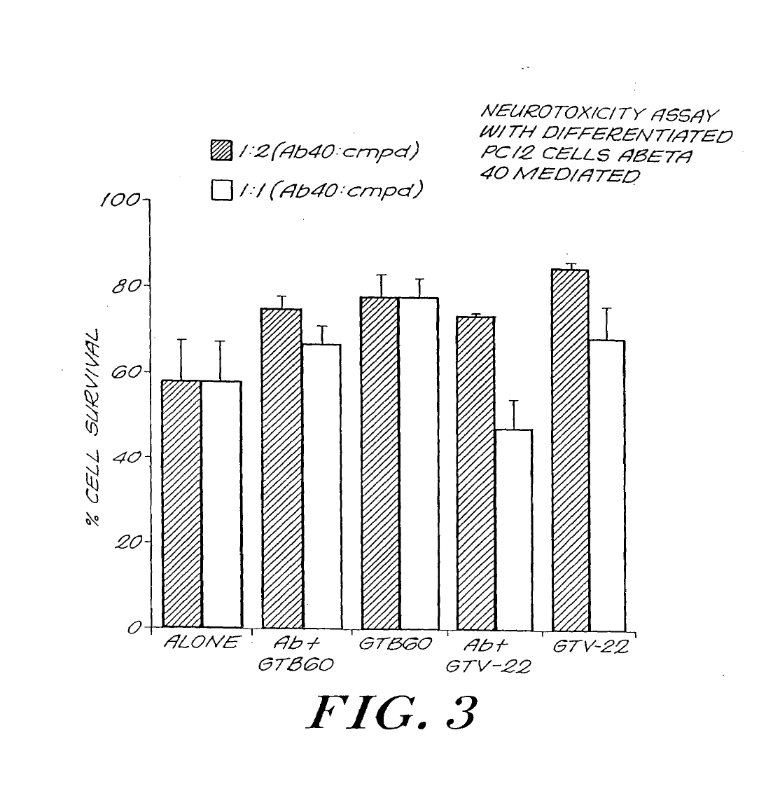

FIG. 4 is a depiction of a bar graph showing the results from an Aβ(1-40) mediated neurotoxicity assay on differentiated PC-12 cells.

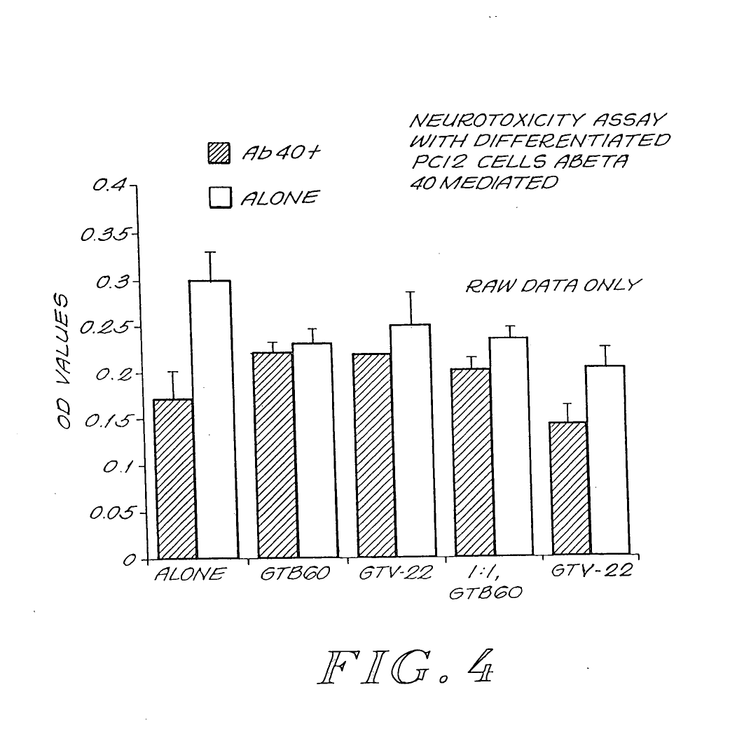

FIG. 5 is a graph illustrating the ability of Aβ to induce neuronal cell death using the SH-5454 neuroblastoma human cell line. Toxicity was measured using 2 different assays: WST-1 assay and 3H-thiperidine uptake.

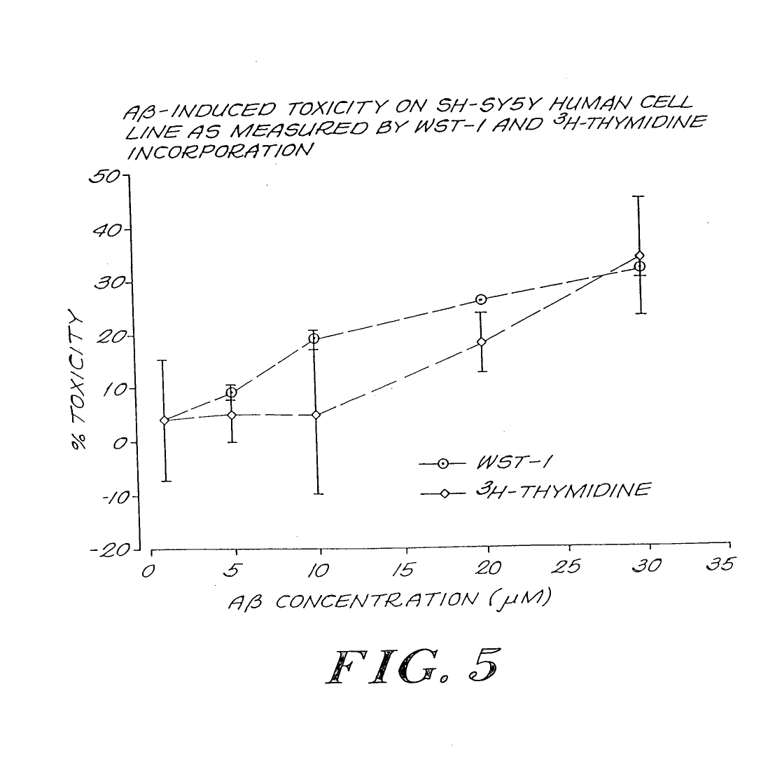

FIG. 6 illustrates the ability of a compound of the present invention, NC-2125 to significantly reduce the Aβ-induced toxicity when incubated at an Aβ: nc-2125 molar ratio of 1:4, laminin, used at an Aβ: laminin molar ratio of 1:10⁻³ is an internal positive control (neuroprotective).

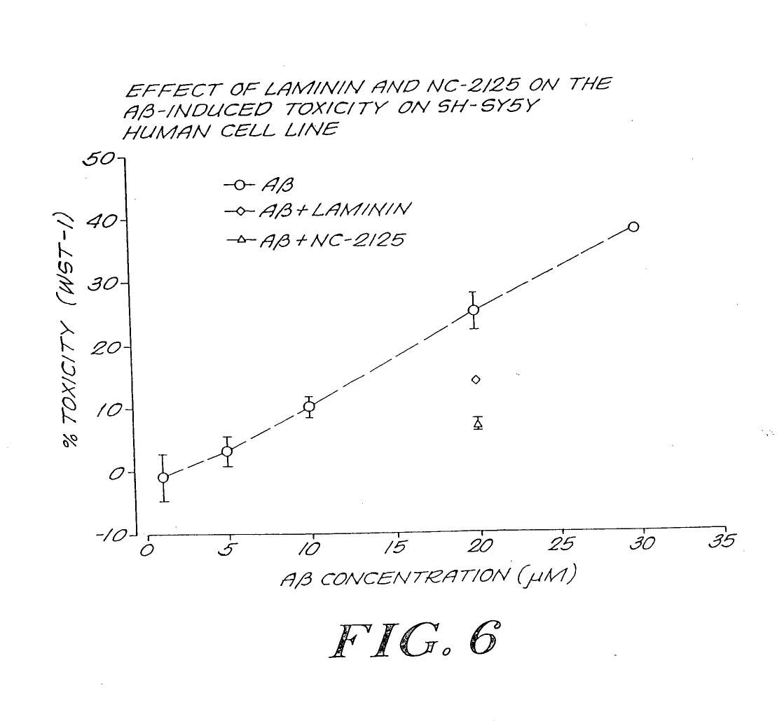

### DETAILED DESCRIPTION OF THE INVENTION

The present invention is based, at least in part, on the discovery that compounds which interfere with the Aβ peptide, e.g., the association of the Aβ peptide, to sites present at the cell surface or to sulfate GAGs, and prevent the triggering of neuronal cell apoptosis or necrosis.

This invention pertains to a method of inhibiting Aβ-induced neuronal cell death. The method includes contacting a neuronal cell with an Aβ-interferer, such that neuronal cell death is inhibited.

As used herein, the language “contacting” is intended to include both in vivo or in vitro methods of bringing an Aβ-interferer or a p75 receptor-interferer into proximity with a neuronal cell, such that the Aβ-interferer or a p75 receptor-interferer can modulate, e.g., inhibit, the death, e.g., apoptosis, of the neuronal cell. For example, the neuronal cell can be contacted with an Aβ-interferer in vivo by administering the Aβ-interferer to a subject either parenterally, e.g., intravenously, intradermally, subcutaneously, orally (e.g., via inhalation), transdermally (topically), transmucosally, or rectally. A neuronal cell can also be conducted in vitro by, for example, adding an Aβ-interferer or a p75 receptor-interferer into a tissue culture dish in which neuronal cells are grown.

The invention further pertains to a method of providing neuroprotection to a subject, comprising administering an Aβ-interferer to the subject, such that neuroprotection is provided.

As used herein, the term “subject” is intended to include animals susceptible to states characterized by neuronal cell death, preferably mammals, most preferably humans. In a preferred embodiment, the subject is a primate. In an even more preferred embodiment, the primate is a human. Other examples of subjects include experimental animals such as mice, rats, dogs, cats, goats, sheep, pigs, and cows. The experimental animal can be an animal model for a disorder, e.g., a transgenic mouse with an Alzheimer's-type neuropathology. A subject can be a human suffering from a neurodegenerative disease, such as Alzheimer's disease, or Parkinson's disease.

As used herein, the term “neuroprotection” is intended to include protection of neuronal cells of a subject from cell death, e.g., cell death induced by an Aβ peptide and/or mediated by an apoptosis related p75 receptor. Neuroprotection includes, for example, inhibition of processes such as the destabilization of the cytoskeleton; the activation of hydrolytic enzymes, such as phospholipase A2, calcium-activated proteases, and calcium-activated endonucleases; the disruption of cell junctions leading to decreased or absent cell-cell communication; and the activation of expression of genes involved in cell death, e.g., immediate-early genes.

### Aβ-Interferers and p75 Receptor-Interferers

In one embodiment, the method of the invention includes contacting a neuronal cell in vitro or administering to a subject in vivo, an effective amount of an Aβ-interferer or a p75 receptor-interferer, which has at least one anionic group covalently attached to a carrier molecule. As used herein, an “Aβ-interferer” refers to a compound which can interfere with the ability of an Aβ-peptide to either form Aβ-fibrils or interact with a cell surface molecule such as a proteoglycan constituent of a basement membrane, e.g. a glycosaminoglycan, a cell surface receptor, e.g., a neurotrophic receptor such as the apoptosis related p75 receptor; or a protein presented by plasma protein, e.g., RAGE. An Aβ-interferer can interfere with the ability of both fibrillar or non-fibrillar Aβ to interact with a cell surface molecule, e.g., the apoptosis related p75 receptor or RAGE. As used herein, a “p75 receptor-interferer” refers to a compound which can interfere with the ability of the apoptosis related p75 receptor to mediate cell death in a neuronal cell. The p75 receptor-interferer can block a ligand binding site on the p75 receptor, it can compete with the natural ligand for binding to the p75 receptor, or it can block the p75 receptor binding site on the natural ligand, thus preventing the ligand-receptor interaction. It should be understood that the description set forth below regarding particular compounds, and formulae is applicable to both examples of Aβ-interferers and P75 receptor-interferers.

The Aβ-interferer or p75 receptor-interferer can have the structure:

Q-[-Y⁻X⁺]ₙ

wherein Y⁻ is an anionic group at physiological pH; Q is a carrier group; X⁺ is a cationic group; and n is an integer. The number of anionic groups (“n”) is selected such that the biodistribution of the Aβ-interferer or p75 receptor-interferer for an intended target site is not prevented while maintaining activity of the Aβ-interferer or p75 receptor-interferer. For example, the number of anionic groups is not so great as to prevent traversal of an anatomical barrier, such as a cell membrane, or entry across a physiological barrier, such as the blood-brain barrier, in situations where such properties are desired. In one embodiment, n is an integer between 1 and 10. In another embodiment, n is an integer between 3 and 8. These compounds are described in U.S. Pat. No. 5,643,562, the contents of which are incorporated herein by reference.

An anionic group of an Aβ-interferer of the invention is a negatively charged moiety that, when attached to a carrier group, can inhibit an Aβ-peptide from either forming Aβ-fibrils or interacting with a cell surface molecule such as a proteoglycan constituent of a basement membrane, e.g. a glycosaminoglycan, a cell surface receptor, e.g., a neurotrophic receptor such as the apoptosis related p75 receptor, or a protein presented by plasma protein, e.g., RAGE, thus preventing neuronal cell death.

An anionic group of a p75 receptor-interferer of the invention is a negatively charged moiety that, when attached to a carrier group, can inhibit the apoptosis related p75 receptor from mediating cell death in a neuronal cell.

For purposes of this invention, the anionic group is negatively charged at physiological pH. Preferably, the anionic Aβ-interferer mimics the structure of a sulfated proteoglycan, i.e., is a sulfated compound or a functional equivalent thereof. “Functional equivalents” of sulfates are intended to include compounds such as sulfamates as well as bioisosteres. Bioisosteres encompass both classical bioisosteric equivalents and non-classical bioisosteric equivalents. Classical and non-classical bioisosteres of sulfate groups are known in the art (see e.g. Silverman, R. B. The Organic Chemistry of Drug Design and Drug Action, Academic Press, Inc.: San Diego, Calif., 1992, pp.19-23). Accordingly, an Aβ-interferer of the invention can comprise at least one anionic group including sulfonates, sulfates, sulfamates, phosphonates, phosphates, carboxylates, and heterocyclic groups of the following formulas:

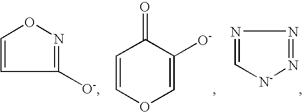

Depending on the carrier group, more than one anionic group can be attached thereto. When more than one anionic group is attached to a carrier group, the multiple anionic groups can be the same structural group (e.g., all sulfonates) or, alternatively, a combination of different anionic groups can be used (e.g., sulfonates, phosphonates, and sulfates, etc.).

The ability of an Aβ-interferer of the invention to inhibit an interaction between an Aβ peptide and a glycoprotein or proteoglycan constituent of a basement membrane can be assessed by an in vitro binding assay, such as the one described in Leveugle B. et al. (1998) J. of Neurochem. 70(2):736-744. Briefly, a constituent of the basement membrane, preferably a glycosaminoglycan (GAG) can be radiolabeled, e.g., at a specific activity of 10,000 cpm, and then incubated with Aβ peptide-Sepharose beads at, for example, a ratio of 5:1 (v/v) in the presence or absence of the Aβ-interferer. The Aβ peptide-Sepharose beads and the radiolabeled GAG can be incubated for approximately 30 minutes at room temperature and then the beads can be successively washed with a Tris buffer solution containing NaCl (0.55 M and 2 M). The binding of the basement membrane constituent (e.g., GAG) to the Aβ-peptide can then be measured by collecting the fractions from the washings and subjecting them to scintillation counting. An Aβ-interferer which inhibits an interaction between an Aβ peptide and a glycoprotein or proteoglycan constituent of a basement membrane, e.g., GAG, will increase the amount of radioactivity detected in the washings.

Preferably, an Aβ-interferer of the invention interacts with a binding site for a basement membrane glycoprotein or proteoglycan in an Aβ peptide and thereby inhibits the binding of the Aβ peptide to the basement membrane constituent, e.g., GAG. Basement membrane glycoproteins and proteoglycans include GAG, laminin, collagen type IV, fibronectin, and heparan sulfate proteoglycan (HSPG). In a preferred embodiment, the therapeutic compound inhibits an interaction between an Aβ peptide and GAG. Consensus binding site motifs for GAG in amyloidogenic proteins have been described (see, for example, Hileman R. E. et al. (1998) BioEssays 20:156-167). For example, a GAG consensus binding motif can be of the general formula X-B-B-X-B-X or X-B-B-B-X-X-B-X, wherein B are basic amino acids (e.g., lysine or arginine) and X are hydropathic amino acids. A GAG consensus binding motif can further be of the general formula T-X-X-B-X-X-T-B-X-X-X-T-B-B, wherein T defines a turn of a basic amino acid, Bs are basic amino acids (e.g., lysine, arginine, or occasionally glutamine) and X are hydropathic amino acids. The distance between the first and the second turn can range from approximately 12 Å to 17 Å. The distance between the second and the third turn can be approximately 14 Å. The distance between the first and the third turn can range from approximately 13 Å to 18 Å. More recently the GAG binding site domain of Aβ (i.e. the 13-16 region: HHQK) has been shown to be responsible for the adherence of Aβ to microglia cell surface leading to its activation (D. Guilian, JBC 1998). These results support the “notion” that interference in the Aβ adherence by blocking its specific GAG binding site will abrogate Aβ neuronal cell death.

Accordingly, in the Aβ-interferers of the invention, when multiple anionic groups are attached to a carrier group, the relative spacing of the anionic groups can be chosen such that the anionic groups (e.g., sulfonates or phosphonates) optimally interact with the basic residues within the GAG binding site (thereby inhibiting interaction of GAG with the site). For example, anionic groups can be spaced approximately 15±1.5 Å, 14±1.5 Å and/or 16±1.5 Å apart, or appropriate multiples thereof, such that the relative spacing of the anionic groups allows for optimal interaction with a binding site for a basement membrane constituent (e.g., GAG) in an Aβ peptide.

Preferably, a p75 receptor-interferer of the invention can block a ligand binding site on the p75 receptor, it can compete with the natural ligand for binding to the p75 receptor, or it can block the p75 receptor binding site on the natural ligand.

An Aβ-interferer or p75 receptor-interferer of the invention typically further comprises a counter cation (i.e., X+in the general formula: Q-[-Y⁻X⁺]ₙ). Cationic groups include positively charged atoms and moieties. If the cationic group is hydrogen, H⁺, then the compound is considered an acid, e.g., ethanesulfonic acid. If hydrogen is replaced by a metal or its equivalent, the compound is a salt of the acid. Pharmaceutically acceptable salts of the Aβ-interferer or p75 receptor-interferer are within the scope of the invention. For example, X⁺ can be a pharmaceutically acceptable alkali metal, alkaline earth, higher valency cation, polycationic counter ion or ammonium. A preferred pharmaceutically acceptable salt is a sodium salt but other salts are also contemplated within their pharmaceutically acceptable range.

Within the Aβ-interferer or p75 receptor-interferer, the anionic group(s) is covalently attached to a carrier group. Suitable carrier groups include aliphatic groups, alicyclic groups, heterocyclic groups, aromatic groups, and groups derived from carbohydrates, polymers, peptides, peptide derivatives, or combinations thereof. A carrier group can be substituted, e.g. with one or more amino, nitro, halogen, thiol or hydroxyl groups.

As used herein, the term “carbohydrate” is intended to include substituted and unsubstituted mono-, oligo-, and polysaccharides. Monosaccharides are simple sugars usually of the formula C₆H₁₂O₆ that can be combined to form oligosaccharides or polysaccharides. Monosaccharides include enantiomers and both the D and L stereoisomers of monosaccharides. Carbohydrates can have multiple anionic groups attached to each monosaccharide moiety. For example, in sucrose octasulfate, four sulfate groups are attached to each of the two monosaccharide moieties.

As used herein, the term “polymer” is intended to include molecules formed by the chemical union of two or more combining subunits called monomers. Monomers are molecules or compounds which usually contain carbon and are of relatively low molecular weight and simple structure. A monomer can be converted to a polymer by combination with itself or other similar molecules or compounds. A polymer may be composed of a single identical repeating subunit or multiple different repeating subunits (copolymers). Polymers within the scope of this invention include substituted and unsubstituted vinyl, acryl, styrene and carbohydrate-derived polymers and copolymers and salts thereof. In one embodiment, the polymer has a molecular weight of approximately 800-1000 Daltons. Examples of polymers with suitable covalently attached anionic groups (e.g., sulfonates or sulfates) include poly(2-acrylamido-2-methyl-1-propanesulfonic acid); poly(2-acrylamido-2-methyl-1-propanesulfonic acid-co-acrylonitrile); poly(2-acrylamido-2-methyl-1-propanesulfonic acid-co-styrene); poly(vinylsulfonic acid); poly(sodium 4-styrenesulfonic acid); and sulfates and/or sulfonates derived from: poly(acrylic acid); poly(methyl acrylate); poly(methyl methacrylate); and poly(vinyl alcohol); and pharmaceutically acceptable salts thereof. Examples of polymers with suitable covalently attached anionic groups include those of the formula:

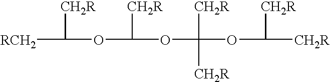

wherein R is SO₃H or OSO₃H; and pharmaceutically acceptable salts thereof.

Peptides and peptide derivatives can also act as carriers. The term “peptide” includes two or more amino acids covalently attached through a peptide bond. Amino acids which can be used in peptide carrier include those naturally occurring amino acids found in proteins such as glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan. The term amino acid further includes analogs, derivatives and congeners of naturally occurring amino acids, one or more of which can be present in a peptide derivative. For example, amino acid analogs can have lengthened or shortened side chains or variant side chains with appropriate functional groups. Also included are the D and L stereoisomers of an amino acid when the structure of the amino acid admits of stereoisomeric forms. The term “peptide derivative” further includes compounds which contain molecules which mimic a peptide backbone but are not amino acids (so-called peptidomimetics), such as benzodiazepine molecules (see e.g. James, G. L. et al. (1993) Science 260:1937-1942). The anionic groups can be attached to a peptide or peptide derivative through a functional group on the side chain of certain amino acids or other suitable functional group. For example, a sulfate group can be attached through the hydroxyl side chain of a serine residue. A peptide can be designed to interact with a binding site for a basement membrane constituent (e.g., a GAG) in an Aβ-peptide (as described above). Accordingly, in one embodiment, the peptide comprises four amino acids and anionic groups (e.g., sulfonates) are attached to the first, second and fourth amino acid. For example, the peptide can be Ser-Ser-Y-Ser, wherein an anionic group is attached to the side chain of each serine residue and Y is any amino acid. In addition to peptides and peptide derivatives, single amino acids can be used as carriers in the Aβ-interferer or p75 receptor-interferer of the invention. For example, cysteic acid, the sulfonate derivative of cysteine, can be used.

The term “aliphatic group” is intended to include organic compounds characterized by straight or branched chains, typically having between 1 and 22 carbon atoms. Aliphatic groups include alkyl groups, alkenyl groups and alkynyl groups. In complex structures, the chains can be branched or cross-linked. Alkyl groups include saturated hydrocarbons having one or more carbon atoms, including straight-chain alkyl groups and branched-chain alkyl groups. Such hydrocarbon moieties may be substituted on one or more carbons with, for example, a halogen, a hydroxyl, a thiol, an amino, an alkoxy, an alkylcarboxy, an alkylthio, or a nitro group. Unless the number of carbons is otherwise specified, “lower aliphatic” as used herein means an aliphatic group, as defined above (e.g., lower alkyl, lower alkenyl, lower alkynyl), but having from one to six carbon atoms. Representatives of such lower aliphatic groups, e.g., lower alkyl groups, are methyl, ethyl, n-propyl, isopropyl, 2-chloropropyl, n-butyl, sec-butyl, 2-aminobutyl, isobutyl, tert-butyl, 3-thiopentyl, and the like. As used herein, the term “amino” means —NH₂; the term “nitro” means —NO₂; the term “halogen” designates —F, —Cl, —Br or —I; the term “thiol” means SH; and the term “hydroxyl” means —OH. Thus, the term “alkylamino” as used herein means —NHR in which R is an alkyl group as defined above. The term “alkylthio” refers to —SR, in which R is an alkyl group as defined above. The term “alkylcarboxyl” as used herein means —COOR, in which R is an alkyl group as defined above. The term “alkoxy” as used herein means —OR, in which R is an alkyl group as defined above. Representative alkoxy groups include methoxy, ethoxy, propoxy, tert-butoxy and the like. The terms “alkenyl” and “alkynyl” refer to unsaturated aliphatic groups analogous to alkyls, but which contain at least one double or triple bond respectively.

The term “alicyclic group” is intended to include closed ring structures of three or more carbon atoms. Alicyclic groups include cycloparaffins or naphthenes which are saturated cyclic hydrocarbons, cycloolefins which are unsaturated with two or more double bonds, and cycloacetylenes which have a triple bond. They do not include aromatic groups. Examples of cycloparaffins include cyclopropane, cyclohexane, and cyclopentane. Examples of cycloolefins include cyclopentadiene and cyclooctatetraene. Alicyclic groups also include fused ring structures and substituted alicyclic groups such as alkyl substituted alicyclic groups. In the instance of the alicyclics such substituents can further comprise a lower alkyl, a lower alkenyl, a lower alkoxy, a lower alkylthio, a lower alkylamino, a lower alkylcarboxyl, a nitro, a hydroxyl, —CF₃, —CN, or the like.

The term “heterocyclic group” is intended to include closed ring structures in which one or more of the atoms in the ring is an element other than carbon, for example, nitrogen, or oxygen. Heterocyclic groups can be saturated or unsaturated and heterocyclic groups such as pyrrole and furan can have aromatic character. They include fused ring structures such as quinoline and isoquinoline. Other examples of heterocyclic groups include pyridine and purine. Heterocyclic groups can also be substituted at one or more constituent atoms with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like.

The term “aromatic group” is intended to include unsaturated cyclic hydrocarbons containing one or more rings. Aromatic groups include 5- and 6-membered single-ring groups which may include from zero to four heteroatoms, for example, benzene, pyrrole, furan, thiophene, imidazole, oxazole, thiazole, triazole, pyrazole, pyridine, pyrazine, pyridazine and pyrimidine, and the like. The aromatic ring may be substituted at one or more ring positions with, for example, a halogen, a lower alkyl, a lower alkenyl, a lower alkoxy, a lower alkylthio, a lower alkylamino, a lower alkylcarboxyl, a nitro, a hydroxyl, —CF₃, —CN, or the like.

In a preferred embodiment of the method of the invention, the Aβ-interferer administered to the subject is comprised of at least one sulfonate group covalently attached to a carrier group, or a pharmaceutically acceptable salt thereof. Accordingly, the an Aβ-interferer or a p75 receptor-interferer can have the structure:

Q-[-SO₃⁻X⁺]ₙ

wherein Q is a carrier group; X⁺ is a cationic group; and n is an integer. Suitable carrier groups and cationic groups are those described hereinbefore. The number of sulfonate groups (“n”) is selected such that the biodistribution of the compound for an intended target site is not prevented while maintaining activity of the compound as discussed earlier. In one embodiment, n is an integer between 1 and 10. In another embodiment, n is an integer between 3 and 8. As described earlier, an Aβ-interferer or a p75 receptor-interferer with multiple sulfonate groups can have the sulfonate groups spaced such that the compound interacts optimally with an HSPG binding site within the Aβ peptide.

In preferred embodiments, the carrier group for a sulfonate(s) is a lower aliphatic group (e.g., a lower alkyl, lower alkenyl or lower alkynyl), a heterocyclic group, and group derived from a disaccharide, a polymer or a peptide or peptide derivative. Furthermore, the carrier can be substituted, e.g. with one or more amino, nitro, halogeno, sulfhydryl or hydroxyl groups. In certain embodiments, the carrier for a sulfonate(s) is an aromatic group.

Examples of suitable sulfonated polymeric Aβ-interferers include poly(2-acrylamido-2-methyl-1-propanesulfonic acid); poly(2-acrylamido-2-methyl- 1-propanesulfonic acid-co-acrylonitrile); poly(2-acrylamido-2-methyl-1-propanesulfonic acid-co-styrene); poly(vinylsulfonic acid); poly(sodium 4-styrenesulfonic acid); a sulfonic acid derivative of poly(acrylic acid); a sulfonic acid derivative of poly(methyl acrylate); a sulfonic acid derivative of poly(methyl methacrylate); and a sulfonate derivative of poly(vinyl alcohol); and pharmaceutically acceptable salts thereof.

A preferred sulfonated polymer is poly(vinylsulfonic acid) (PVS) or a pharmaceutically acceptable salt thereof, preferably the sodium salt thereof. In one embodiment, PVS having a molecular weight of about 800-1000 Daltons is used. PVS may be used as a mixture of stereoisomers or as a single active isomer.

Preferred sulfonated saccharides include 5-deoxy-1,2-O-isopropylidene-α-D-xylofuranose-5-sulfonic acid (XXIII, shown as the sodium salt).

Preferred lower aliphatic sulfonated Aβ-interferers for use in the invention include ethanesulfonic acid; 2-aminoethanesulfonic acid (taurine); cysteic acid (3-sulfoalanine or α-amino-β-sulfopropionic acid); 1-propanesulfonic acid; 1,2-ethanedisulfonic acid; 1,3-propanedisulfonic acid; 1,4-butanedisulfonic acid; 1,5-pentanedisulfonic acid; and 4-hydroxybutane-1-sulfonic acid (VIII, shown as the sodium salt); and pharmaceutically acceptable salts thereof. Other aliphatic sulfonated Aβ-interferers contemplated for use in the invention include 1-butanesulfonic acid (XLVII, shown as the sodium salt), 2-propanesulfonic acid (XLIX, shown as the sodium salt), 3-pentanesulfonic acid (L, shown as the sodium salt), 4-heptanesulfonic acid (LII, shown as the sodium salt), 1-decanesulfonic acid (XLVIII, shown as the sodium salt); and pharmaceutically acceptable salts thereof. Sulfonated substituted aliphatic Aβ-interferers contemplated for use in the invention include 3-amino-1-propanesulfonic acid (XXII, shown as the sodium salt), 3-hydroxy-1-propanesulfonic acid sulfate (XXXV, shown as the disodium salt), 1,7-dihydroxy-4-heptanesulfonic acid (LIII, shown as the sodium salt); and pharmaceutically acceptable salts thereof. Yet other sulfonated compounds contemplated for use in the invention include 2-[(4-pyridinyl)amido]ethanesulfonic acid (LIV, depicted as the sodium salt), and pharmaceutically acceptable salts thereof.

Preferred heterocyclic sulfonated Aβ-interferers include 3-(N-morpholino)-1-propanesulfonic acid; and tetrahydrothiophene-1,1-dioxide-3,4-disulfonic acid; and pharmaceutically acceptable salts thereof.

Aromatic sulfonated Aβ-interferers include 1,3-benzenedisulfonic acid (XXXVI, shown as the disodium salt), 2,5-dimethoxy-1,4-benzenedisulfonic acid (depicted as the disodium salt, XXXVII, or the dipotassium salt, XXXIX), 4-amino-3-hydroxy-1-naphthalenesulfonic acid (XLIII), 3,4-diamino-1-naphthalenesulfonic acid (XLIV); and pharmaceutically acceptable salts thereof.

In another embodiment of the method of the invention, the Aβ-interferer administered to the subject is comprised of at least one sulfate group covalently attached to a carrier group, or a pharmaceutically acceptable salt thereof. Accordingly, the Aβ-interferer or the p75 receptor-interferer can have the structure:

Q-[-OSO₃⁻X⁺]ₙ

wherein Q is a carrier group; X⁺ is a cationic group; and n is an integer. Suitable carriers and cationic groups are those described hereinbefore. The number of sulfate groups (“n”) is selected such that the biodistribution of the compound for an intended target site is not prevented while maintaining activity of the Aβ-interferer as discussed earlier. In one embodiment, n is an integer between 1 and 10. In another embodiment, n is an integer between 3 and 8. As described earlier, an Aβ-interferer with multiple sulfate groups can have the sulfate groups spaced such that the compound interacts optimally with a GAG binding site within an Aβ peptide.

In preferred embodiments, the carrier group for a sulfate(s) is a lower aliphatic group (e.g., a lower alkyl, lower alkenyl or lower alkynyl), an aromatic group, a group derived from a disaccharide, a polymer or a peptide or peptide derivative. Furthermore, the carrier can be substituted, e.g. with one or more amino, nitro, halogeno, sulfhydryl or hydroxyl groups.

Examples of suitable sulfated polymeric Aβ-interferers or p75 receptor-interferers include poly(2-acrylamido-2-methyl-propyl sulfuric acid); poly(2-acrylamido-2-methyl-propyl sulfuric acid-co-acrylonitrile); poly(2-acrylamido-2-methyl-propyl sulfuric acid-co-styrene); poly(vinylsulfuric acid); poly(sodium4-styrenesulfate); a sulfate derivative of poly(acrylic acid); a sulfate derivative of poly(methyl acrylate); a sulfate derivative of poly(methyl methacrylate); and a sulfate derivative of poly(vinyl alcohol); and pharmaceutically acceptable salts thereof.

A preferred sulfated polymer is poly(vinylsulfuric acid) or pharmaceutically acceptable salt thereof.

A preferred sulfated disaccharide is sucrose octasulfate or pharmaceutically acceptable salt thereof. Other sulfated saccharides contemplated for use in the invention include the acid form of methyl-α-D-glucopyranoside 2,3-disulfate (XVI), methyl 4,6-O-benzylidene-α-D-glucopyranoside 2,3-disulfate (XVII), 2,3,4,3′,4′-sucrose pentasulfate (XXXIII), 1,3:4,6-di-O-benzylidene-D-mannitol 2,5-disulfate (XLI), D-mannitol 2,5-disulfate (XLII), 2,5-di-O-benzyl-D-mannitol tetrasulfate (XLV); and pharmaceutically acceptable salts thereof.

Preferred lower aliphatic sulfated Aβ-interferers for use in the invention include ethyl sulfuric acid; 2-aminoethan-1-ol sulfuric acid; 1-propanol sulfuric acid; 1,2-ethanediol disulfuric acid; 1,3-propanediol disulfuric acid; 1,4-butanediol disulfuric acid; 1,5-pentanediol disulfuric acid; and 1,4-butanediol monosulfuric acid; and pharmaceutically acceptable salts thereof. Other sulfated aliphatic Aβ-interferers contemplated for use in the invention include the acid form of 1,3-cyclohexanediol disulfate (XL), 1,3,5-heptanetriol trisulfate (XIX), 2-hydroxymethyl-1,3-propanediol trisulfate (XX), 2-hydroxymethyl-2-methyl-1,3-propanediol trisulfate (XXI), 1,3,5,7-heptanetetraol tetrasulfate (XLVI), 1,3,5,7,9-nonane pentasulfate (LI); and pharmaceutically acceptable salts thereof. Other sulfated Aβ-interferers contemplated for use in the invention include the acid form of 2-amino-2-hydroxymethyl-1,3-propanediol trisulfate (XXIV), 2-benzyloxy-1,3-propanediol disulfate (XXIX), 3-hydroxypropylsulfamic acid sulfate (XXX)2,2′-iminoethanol disulfate (XXXI), N,N-bis(2-hydroxyethyl)sulfamic acid disulfate (XXXII); and pharmaceutically acceptable salts thereof.

Preferred heterocyclic sulfated Aβ-interferers include 3-(N-morpholino)-1-propyl sulfuric acid; and tetrahydrothiophene-3,4-diol-1,1-dioxide disulfuric acid; and pharmaceutically acceptable salts thereof.

The invention further contemplates the use of prodrugs which are converted in vivo to the Aβ-interferers used in the methods of the invention (see, e.g., R. B. Silverman, 1992, “The Organic Chemistry of Drug Design and Drug Action”, Academic Press, Chp. 8). Such prodrugs can be used to alter the biodistribution (e.g., to allow compounds which would not typically cross the blood-brain barrier to cross the blood-brain barrier) or the pharmacokinetics of the Aβ-interferer. For example, an anionic group, e.g., a sulfate or sulfonate, can be esterified, e.g, with a methyl group or a phenyl group, to yield a sulfate or sulfonate ester. When the sulfate or sulfonate ester is administered to a subject, the ester is cleaved, enzymatically or non-enzymatically, reductively or hydrolytically, to reveal the anionic group. Such an ester can be cyclic, e.g., a cyclic sulfate or sultone, or two or more anionic moieties may be esterified through a linking group. Exemplary cyclic Aβ-interferers include, for example, 2-sulfobenzoic acid cyclic anhydride (LV), 1,3-propane sultone (LVI), 1,4-butane sultone (LVII), 1,3-butanediol cyclic sulfate (LVIII), α-chloro-α-hydroxy-o-toluenesulfonic acid γ-sultone (LIX), and 6-nitronaphth-[1,8-cd]-1,2,-oxathiole 2,2-dioxide (LX). In a preferred embodiment, the prodrug is a cyclic sulfate or sultone. An anionic group can be esterified with moieties (e.g., acyloxymethyl esters) which are cleaved to reveal an intermediate Aβ-interferer which subsequently decomposes to yield the active Aβ-interferer. In another embodiment, the prodrug is a reduced form of a sulfate or sulfonate, e.g., a thiol, which is oxidized in vivo to the Aβ-interferer. Furthermore, an anionic moiety can be esterified to a group which is actively transported in vivo, or which is selectively taken up by target organs. The ester can be selected to allow specific targeting of the Aβ-interferers to particular organs, as described below for carrier moieties.

Carrier groups useful in the Aβ-interferers include groups previously described, e.g. aliphatic groups, alicyclic groups, heterocyclic groups, aromatic groups, groups derived from carbohydrates, polymers, peptides, peptide derivatives, or combinations thereof. Suitable polymers include substituted and unsubstituted vinyl, acryl, styrene and carbohydrate-derived polymers and copolymers and salts thereof. Preferred carrier groups include a lower alkyl group, a heterocyclic group, a group derived from a disaccharide, a polymer, a peptide, or peptide derivative.

Carrier groups useful in the present invention may also include moieties which allow the Aβ-interferer to be selectively delivered to a target organ or organs. For example, if delivery of a tAβ-interferer to the brain is desired, the carrier group may include a moiety capable of targeting the Aβ-interferer to the brain, by either active or passive transport (a “targeting moiety”). Illustratively, the carrier group may include a redox moiety, as described in, for example, U.S. Pat. Nos. 4,540,564 and 5,389,623, both to Bodor. These patents disclose drugs linked to dihydropyridine moieties which can enter the brain, where they are oxidized to a charged pyridinium species which is trapped in the brain. Thus, drug accumulates in the brain. Exemplary pyridine/dihydropyridine compounds of the invention include sodium 2-(nicotinylamido)-ethanesulfonate (LXII), and 1-(3-sulfopropyl)-pyridinium betaine (LXIII). Other carrier moieties include groups, such as those derived from amino acids or thyroxine, which can be passively or actively transported in vivo. An illustrative compound is phenylalanyltaurine (LXIX), in which a taurine molecule is conjugated to a phenylalanine (a large neutral amino acid). Such a carrier moiety can be metabolically removed in vivo, or can remain intact as part of an active Aβ-interferer. Structural mimics of amino acids (and other actively transported moieties) are also useful in the invention (e.g., 1-(aminomethyl)-1-(sulfomethyl)-cyclohexane (LXX)). Other exemplary amino acid mimetics include p-(sulfomethyl)phenylalanine (LXXII), p-(1,3-disulfoprop-2-yl)phenylalanine (LXXIII), and O-(1,3-disulfoprop-2-yl)tyrosine (LXXIV). Exemplary thyroxine mimetics include compounds LXXV, LXVI, and LXXVII. Many targeting moieties are known, and include, for example, asialoglycoproteins (see, e.g. Wu, U.S. Pat. No. 5,166,320) and other ligands which are transported into cells via receptor-mediated endocytosis (see below for further examples of targeting moieties which may be covalently or non-covalently bound to a carrier molecule). Furthermore, the Aβ-interferers of the invention may bind to amyloidogenic proteins, e.g., Aβ peptide, in the circulation and thus be transported to the site of action.

The targeting and prodrug strategies described above can be combined to produce an Aβ-interferer that can be transported as a prodrug to a desired site of action and then unmasked to reveal an active Aβ-interferer. For example, the dihydropyrine strategy of Bodor (see supra) can be combined with a cyclic prodrug, as for example in the compound 2-(1-methyl-1,4-dihydronicotinyl)amidomethyl-propanesultone (LXXI).

In one embodiment, the Aβ-interferer in the pharmaceutical compositions is a sulfonated polymer, for example poly(2-acrylamido-2-methyl-1-propanesulfonic acid); poly(2-acrylamido-2-methyl-1-propanesulfonic acid-co-acrylonitrile); poly(2-acrylamido-2-methyl-1-propanesulfonic acid-co-styrene); poly(vinylsulfonic acid); poly(sodium 4-styrenesulfonic acid); a sulfonate derivative of poly(acrylic acid); a sulfonate derivative of poly(methyl acrylate); a sulfonate derivative of poly(methyl methacrylate); and a sulfonate derivative of poly(vinyl alcohol); and pharmaceutically acceptable salts thereof.

In another embodiment, the Aβ-interferer in the pharmaceutical compositions is a sulfated polymer, for example poly(2-acrylamido-2-methyl-1-propyl sulfuric acid); poly(2-acrylamido-2-methyl-1-propyl sulfuric acid-co-acrylonitrile); poly(2-acrylamido-2-methyl-1-propyl sulfuric acid-co-styrene); poly(vinyl sulfuric acid); poly(sodium 4-styrenesulfate); a sulfate derivative of poly(acrylic acid); a sulfate derivative of poly(methyl acrylate); a sulfate derivative of poly(methyl methacrylate); and pharmaceutically acceptable salts thereof.

The Aβ-interferer or p75 receptor-interferer can also have the structure:

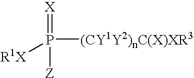

in which Z is XR² or R⁴, R¹ and R² are each independently hydrogen, a substituted or unsubstituted aliphatic group (preferably a branched or straight-chain aliphatic moiety having from 1 to 24 carbon atoms in the chain; or an unsubstituted or substituted cyclic aliphatic moiety having from 4 to 7 carbon atoms in the aliphatic ring; preferred aliphatic and cyclic aliphatic groups are alkyl groups, more preferably lower alkyl), an aryl group, a heterocyclic group, or a salt-forming cation; R³ is hydrogen, lower alkyl, aryl, or a salt-forming cation; X is, independently for each occurrence, O or S; R⁴ is hydrogen, lower alkyl, aryl or amino; Y¹ and Y² are each independently hydrogen, halogen (e.g., F, Cl, Br, or I), lower alkyl, amino (including alkylamino, dialkylamino, arylamino, diarylamino, and alkylarylamino), hydroxy, alkoxy; or aryloxy; and n is an integer from 0 to 12 (more preferably 0 to 6, more preferably 0 or 1); such that amyloid deposition is modulated. These compounds are described in U.S. application Ser. No. 08/912,574, the contents of which are incorporated herein by reference.

Preferred Aβ-interferers or p75 receptor-interferers for use in the invention include compounds in which both R¹ and R² are pharmaceutically acceptable salt-forming cations. It will be appreciated that the stoichiometry of an anionic compound to a salt-forming counterion (if any) will vary depending on the charge of the anionic portion of the compound (if any) and the charge of the counterion. In a particularly preferred embodiment, R¹, R² and R³ are each independently a sodium, potassium or calcium cation. In certain embodiments in which at least one of R¹ and R² is an aliphatic group, the aliphatic group has between 1 and 10 carbons atoms in the straight or branched chain, and is more preferably a lower alkyl group. In other embodiments in which at least one of R¹ and R² is an aliphatic group, the aliphatic group has between 10 and 24 carbons atoms in the straight or branched chain. In certain preferred embodiments, n is 0 or 1; more preferably, n is 0. In certain preferred embodiments of the therapeutic compounds, Y¹ and Y² are each hydrogen.

In certain preferred embodiments, the Aβ-interferer or p75 receptor-interferer of the invention can have the structure:

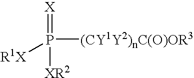

in which R¹, R², R³, Y¹, Y², X and n are as defined above. In more preferred embodiments, the Aβ-interferer or p75 receptor-interferer of the invention can have the structure:

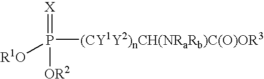

in which R¹, R², R³, Y¹, Y², and X are as defined above, Rₐ and R_{b} are each independently hydrogen, alkyl, aryl, or heterocyclyl, or Rₐ and R_{b}, taken together with the nitrogen atom to which they are attached, form a cyclic moiety having from 3 to 8, atoms in the ring, and n is an integer from 0 to 6. In certain preferred embodiments, Rₐ and R_{b} are each hydrogen. In certain preferred embodiments, a compound of the invention comprises an α-amino acid (or α-amino acid ester), more preferably a L-α-amino acid or ester.

The Z, R¹, R², R³, Y¹, Y² and X groups are each independently selected such that the biodistribution of the Aβ-interferer or p75 receptor-interferer for an intended target site is not prevented while maintaining activity of the Aβ-interferer or p75 receptor-interferer. For example, the number of anionic groups (and the overall charge on the therapeutic compound) should not be so great as to prevent traversal of an anatomical barrier, such as a cell membrane, or entry across a physiological barrier, such as the blood-brain barrier, in situations where such properties are desired. For example, it has been reported that esters of phosphonoformate have biodistribution properties different from, and in some cases superior to, the biodistribution properties of phosphonoformate (see, e.g., U.S. Pat. Nos. 4,386,081 and 4,591,583 to Helgstrand et al., and U.S. Pat. Nos. 5,194,654 and 5,463,092 to Hostetler et al.). Thus, in certain embodiments, at least one of R¹ and R² is an aliphatic group (more preferably an alkyl group), in which the aliphatic group has between 10 and 24 carbons atoms in the straight or branched chain. The number, length, and degree of branching of the aliphatic chains can be selected to provide a desired characteristic, e.g., lipophilicity. In other embodiments, at least one of R¹ and R² is an aliphatic group (more preferably an alkyl group), in which the aliphatic group has between 1 and 10 carbons atoms in the straight or branched chain. Again, the number, length, and degree of branching of the aliphatic chains can be selected to provide a desired characteristic, e.g., lipophilicity or ease of ester cleavage by enzymes. In certain embodiments, a preferred aliphatic group is an ethyl group.

In another embodiment, the Aβ-interferer or p75 receptor-interferer of the invention can have the structure:

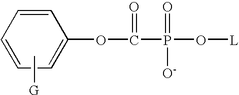

in which G represents hydrogen or one or more substituents on the aryl ring (e.g., alkyl, aryl, halogen, amino, and the like) and L is a substituted alkyl group (in certain embodiments, preferably a lower alkyl), more preferably a hydroxy-substituted alkyl or an alkyl substituted with a nucleoside base. In certain embodiments, G is hydrogen or an electron-donating group. In embodiments in which G is an electron-withdrawing group, G is preferably an electron withdrawing group at the meta position. The term “electron-withdrawing group” is known in the art, and, as used herein, refers to a group which has a greater electron-withdrawing than hydrogen. A variety of electron-withdrawing groups are known, and include halogens (e.g., fluoro, chloro, bromo, and iodo groups), nitro, cyano, and the like. Similarly, the term “electron-donating group”, as used herein, refers to a group which is less electron-withdrawing than hydrogen. In embodiments in which G is an electron donating group, G can be in the ortho, meta or para position.

In certain preferred embodiments, L is a moiety selected from the group consisting of:

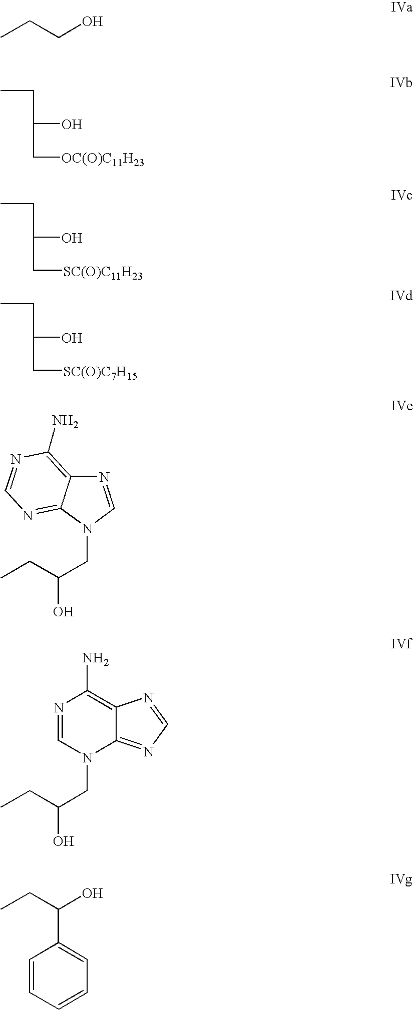

Table 1 lists data pertinent to the characterization of these compounds using art-recognized techniques. The compounds IVa-IVg in Table 1 are corresponding to the following structure, in which L is a group selected from the above-listed (Groups IVa-IVg) with the same number.

TABLE 1

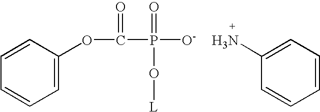

| COM- | | | FAB- |
|---|---|---|---|
| POUND | ³¹P NMR | ¹³C NMR | MS(−) |
| IVa | −6.33 | 60.97 CH₂OH (d, J = 6 Hz) | 245.2 |
| | (DMSO-d₆) | 66.76 CHOH (d, J = 7.8 Hz) | |
| | | 121.65, 121.78, 121.99, 125.71, | |
| | | 129.48, 129.57, 126.43 | |
| | | Aromatic CH | |
| | | 134.38 Aniline C—N | |
| | | 150.39 Phenyl C—O (d, J = 7 Hz) | |
| | | 171.57 P—C═O (d, J = 234Hz) | |
| IVb | −6.41 | 13.94 CH₃ | 456 |
| | (DMSO-d₆) | 22.11, 24.40, 28.56, 28.72, 28.99, | |
| | | 29.00, 31.30, 33.43, —(CH₂)₁₀— | |
| | | 65.03 CH₂—OC(O) | |
| | | 66.60 CH₂—OP (d, J = 5.6 Hz) | |
| | | 67.71 CH2—OH (d, J = 6 Hz) | |
| | | 121.73, 121.10, 125.64, 126.57, | |
| | | 129.40, 129.95, Aromatic CH | |
| | | 134.04 Aniline C—N | |
| | | 150.31 Phenyl C—O | |
| | | 171.44 P—C═O (d, J = 6.7 Hz) | |
| | | 172.83 O—C═O | |
| IVc | −6.46 | 13.94 CH₃ | 471 |
| | (DMSO-d₆) | 22.11, 25.10, 28.68, 28.72, | |
| | | 28.85, 29.00, 30.76, 31.31, 32.10, | |
| | | —(CH₂)₁₀— | |
| | | 43.36 CH₂—S | |
| | | 68.43 CH₂—OH | |
| | | 68.43 CH—OH (d, J = 6.3 Hz) | |
| | | 68.76 P—O—CH₂-9d, J = 5.8 Hz) | |
| | | 121.75, 122.03, 125.62, 126.37, | |
| | | 129.30, 129.53, Aromatic CH | |
| | | 134.23 Aniline C—N | |
| | | 150.37 Phenyl C—O (d, J = 6.7 Hz) | |
| | | 171.47 P—C═O (d, J = 234.0 Hz) | |
| | | 198.47 S—C═O | |
| IVd | −6.61 | 13.94 CH₃ | 416 |
| | (DMSO-d₆) | 22.06, 25.14, 28.24, 28.35, | |
| | | 31.09, 32.14 | |
| | | —CH₂)₆— | |
| | | 43.40 CH₂—S | |
| | | 68.50 P—O—CH₂— (d, J = 5.8 Hz) | |

An anionic group (i.e., a phosphonate or carboxylate group) of an Aβ-interferer or a p75 receptor-interferer of the invention is a negatively charged moiety that, in certain preferred embodiments, can modulate interaction between an Aβ-peptide and a component of a basement membrane, e.g., GAG or the p75 receptor, to, for example, modulate the formation of Aβ-fibrils or cell death.

It will be noted that the structure of some of the Aβ-interferers or p75 receptor-interferers of this invention includes asymmetric carbon atoms. It is to be understood accordingly that the isomers (e.g., enantiomers and diastereomers) arising from such asymmetry are included within the scope of this invention. Such isomers can be obtained in substantially pure form by classical separation techniques and by sterically controlled synthesis. For the purposes of this application, unless expressly noted to the contrary, an Aβ-interferer or a p75 receptor-interferer shall be construed to include both the R or S stereoisomers at each chiral center.

In certain embodiments, an Aβ-interferer or a p75 receptor-interferer of the invention comprises a cation (i.e., in certain embodiments, at least one of R¹, R² or R³ is a cation). If the cationic group is hydrogen, H⁺, then the Aβ-interferer or p75 receptor-interferer is considered an acid, e.g., phosphonoformic acid. If hydrogen is replaced by a metal ion or its equivalent, the Aβ-interferer or p75 receptor-interferer is a salt of the acid. Pharmaceutically acceptable salts of the Aβ-interferer or p75 receptor-interferer are within the scope of the invention. For example, at least one of R¹, R² or R³ can be a pharmaceutically acceptable alkali metal (e.g., Li, Na, or K), ammonium cation, alkaline earth cation (e.g., Ca²⁺, Ba²⁺, Mg²⁺), higher valency cation, or polycationic counter ion (e.g., a polyammonium cation). (See, e.g., Berge et al. (1977) “Pharmaceutical Salts”, J. Pharm. Sci. 66:1-19). It will be appreciated that the stoichiometry of an anionic compound to a salt-forming counterion (if any) will vary depending on the charge of the anionic portion of the compound (if any) and the charge of the counterion. Preferred pharmaceutically acceptable salts include a sodium, potassium or calcium salt, but other salts are also contemplated within their pharmaceutically acceptable range.

The term “pharmaceutically acceptable esters” refers to the relatively non-toxic, esterified products of the Aβ-interferers or p75 receptor-interferers of the present invention. These esters can be prepared in situ during the final isolation and purification of the Aβ-interferers or p75 receptor-interferers or by separately reacting the purified Aβ-interferer or p75 receptor-interferer in its free acid form or hydroxyl with a suitable esterifying agent; either of which are methods known to those skilled in the art. Carboxylic acids and phosphonic acids can be converted into esters according to methods well known to one of ordinary skill in the art, e.g., via treatment with an alcohol in the presence of a catalyst. A preferred ester group (e.g., when R³ is lower alkyl) is an ethyl ester group.

The term “alkyl” refers to the saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 4-10 carbon atoms in their ring structure, and more preferably have 4-7 carbon atoms in the ring structure. The term “lower alkyl” refers to alkyl groups having from 1 to 6 carbons in the chain, and to cycloalkyls having from 3 to 6 carbons in the ring structure.

Moreover, the term “alkyl” (including “lower alkyl”) as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfate, sulfonato, sulfamoyl, sulfonamido, nitro, trifluoromethyl, cyano, azido, heterocyclyl, aralkyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (benzyl)).

The term “alkoxy”, as used herein, refers to a moiety having the structure —O-alkyl, in which the alkyl moiety is described above.

The term “aryl” as used herein includes 5- and 6-membered single-ring aromatic groups that may include from zero to four heteroatoms, for example, unsubstituted or substituted benzene, pyrrole, furan, thiophene, imidazole, oxazole, thiazole, triazole, pyrazole, pyridine, pyrazine, pyridazine and pyrimidine, and the like. Aryl groups also include polycyclic fused aromatic groups such as naphthyl, quinolyl, indolyl, and the like. The aromatic ring can be substituted at one or more ring positions with such substituents, e.g., as described above for alkyl groups. Preferred aryl groups include unsubstituted and substituted phenyl groups.

The term “aryloxy”, as used herein, refers to a group having the structure —O-aryl, in which the aryl moiety is as defined above.

The term “amino,” as used herein, refers to an unsubstituted or substituted moiety of the formula —NRₐR_{b}, in which Rₐ and R_{b} are each independently hydrogen, alkyl, aryl, or heterocyclyl, or Rₐ and R_{b}, taken together with the nitrogen atom to which they are attached, form a cyclic moiety having from 3 to 8 atoms in the ring. Thus, the term “amino” is intended to include cyclic amino moieties such as piperidinyl or pyrrolidinyl groups, unless otherwise stated. An “amino-substituted amino group” refers to an amino group in which at least one of Rₐ and R_{b}, is further substituted with an amino group.

In a preferred embodiment, R¹ or R² can be (for at least one occurrence) a long-chain aliphatic moiety. The term “long-chain aliphatic moiety” as used herein, refers to a moiety having a straight or branched chain aliphatic moiety (e.g., an alkyl or alkenyl moiety) having from 10 to 24 carbons in the aliphatic chain, e.g., the long-chain aliphatic moiety is an aliphatic chain of a fatty acid (preferably a naturally-occurring fatty acid). Representative long-chain aliphatic moieties include the aliphatic chains of stearic acid, oleic acid, linolenic acid, and the like.

In certain embodiments, the Aβ-interferer or p75 receptor-interferer of the invention can have the structure:

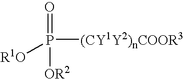

in which R¹ and R² are each independently hydrogen, an aliphatic group (preferably a branched or straight-chain aliphatic moiety having from 1 to 24 carbon atoms, more preferably 10-24 carbon atoms, in the chain; or an unsubstituted or substituted cyclic aliphatic moiety having from 4 to 7 carbon atoms in the aliphatic ring), an aryl group, a heterocyclic group, or a salt-forming cation; R³ is hydrogen, lower alkyl, aryl, or a salt-forming cation; Y¹ and Y² are each independently hydrogen, halogen (e.g., F, Cl, Br, or I), lower alkyl, hydroxy, alkoxy, or aryloxy; and n is an integer from 0 to 12; such that amyloid deposition is modulated. In one preferred embodiment, Aβ-interferers or p75 receptor-interferers of the invention prevent or inhibit amyloid deposition in a subject to which the Aβ-interferer or p75 receptor-interferer is administered. Preferred Aβ-interferers or p75 receptor-interferers for use in the invention include compounds in which both R¹ and R² are pharmaceutically acceptable salt-forming cations. In a particularly preferred embodiment, R¹, R² and R³ are each independently a sodium, potassium or calcium cation, and n is 0. In certain preferred embodiments of the therapeutic compounds, Y¹ and Y² are each hydrogen. Particularly preferred Aβ-interferers or p75 receptor-interferers are salts of phosphonoformate. Trisodium phosphonoformate (foscarnet sodium or Foscavir®) is commercially available (e.g., from Astra), and its clinical pharmacology has been investigated (see, e.g., “Physician's Desk Reference”, 51st Ed., pp. 541-545 (1997)).

In another embodiment, the Aβ-interferer or p75 receptor-interferer used in the invention can be an aminophosphonate, a bisphosphonate, a phosphonocarboxylate derivative, a phosphonate derivative, or a phosphono carbohydrate. For example, the Aβ-interferer or p75 receptor-interferer can be one of the compounds described in Appendix A submitted herewith.

### Pharmaceutically Acceptable Formulations

In the method of the invention, the Aβ-interferer or p75 receptor-interferer can be administered in a pharmaceutically acceptable formulation. The present invention pertains to any pharmaceutically acceptable formulations, such as synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes.

In one embodiment, the pharmaceutically acceptable formulations comprise a polymeric matrix.

The terms “polymer” or “polymeric” are art-recognized and include a structural framework comprised of repeating monomer units which. is capable of delivering an Aβ-interferer or a p75 receptor-interferer, such that treatment of a targeted condition, e.g., a CNS injury, occurs. The terms also include co-polymers and homopolymers e.g., synthetic or naturally occurring. Linear polymers, branched polymers, and cross-linked polymers are also meant to be included.

For example, polymeric materials suitable for forming the pharmaceutically acceptable formulation employed in the present invention, include naturally derived polymers such as albumin, alginate, cellulose derivatives, collagen, fibrin, gelatin, and polysaccharides, as well as synthetic polymers such as polyesters (PLA, PLGA), polyethylene glycol, poloxomers, polyanhydrides, and pluronics. These polymers are biocompatible with the nervous system, including the central nervous system, they are biodegradable within the central nervous system without producing any toxic byproducts of degradation, and they possess the ability to modify the manner and duration of Aβ-interferer or p75 receptor-interferer release by manipulating the polymer's kinetic characteristics. As used herein, the term “biodegradable” means that the polymer will degrade over time by the action of enzymes, by hydrolytic action and/or by other similar mechanisms in the body of the subject. As used herein, the term “biocompatible” means that the polymer is compatible with a living tissue or a living organism by not being toxic or injurious and by not causing an immunological rejection.

Polymers can be prepared using methods known in the art (Sandler, S. R.; Karo, W. Polymer Syntheses; Harcourt Brace: Boston, 1994; Shalaby, W.; Ikada, Y.; Langer, R.; Williams, J. Polymers of Biological and Biomedical Significance (ACS Symposium Series 540; American Chemical Society: Washington, D.C., 1994). Polymers can be designed to be flexible; the distance between the bioactive side-chains and the length of a linker between the polymer backbone and the group can be controlled. Other suitable polymers and methods for their preparation are described in U.S. Pat. Nos. 5,455,044 and 5,576,018, the contents of which are incorporated herein by reference.

The polymeric formulations are preferably formed by dispersion of the Aβ-interferer or p75 receptor-interferer within liquefied polymer, as described in U.S. Pat. No. 4,883,666, the teachings of which are incorporated herein by reference, or by such methods as bulk polymerization, interfacial polymerization, solution polymerization and ring polymerization as described in Odian G., Principles of Polymerization and ring opening polymerization, 2nd ed., John Wiley & Sons, New York, 1981, the contents of which are incorporated herein by reference. The properties and characteristics of the formulations are controlled by varying such parameters as the reaction temperature, concentrations of polymer and Aβ-interferer or p75 receptor-interferer, types of solvent used, and reaction times.

In addition to the Aβ-interferer or p75 receptor-interferer and the pharmaceutically acceptable polymer, the pharmaceutically acceptable formulation used in the method of the invention can comprise additional pharmaceutically acceptable carriers and/or excipients. As used herein, “pharmaceutically acceptable carrier” includes any and all solvents, dispersion media, coatings, antibacterial and anti fungal agents, isotonic and absorption delaying agents, and the like that are physiologically compatible. For example, the carrier can be suitable for injection into the cerebrospinal fluid. Excipients include pharmaceutically acceptable stabilizers and disintegrants.

The Aβ-interferer or p75 receptor-interferer can be encapsulated in one or more pharmaceutically acceptable polymers, to form a microcapsule, microsphere, or microparticle, terms used herein interchangeably. Microcapsules, microspheres, and microparticles are conventionally free-flowing powders consisting of spherical particles of 2 millimeters or less in diameter, usually 500 microns or less in diameter. Particles less than 1 micron are conventionally referred to as nanocapsules, nanoparticles or nanospheres. For the most part, the difference between a microcapsule and a nanocapsule, a microsphere and a nanosphere, or microparticle and nanoparticle is size; generally there is little, if any, difference between the internal structure of the two. In one aspect of the present invention, the mean average diameter is less than about 45 μm, preferably less than 20 μm, and more preferably between about 0.1 and 10 μm.

In another embodiment, the pharmaceutically acceptable formulations comprise lipid-based formulations. Any of the known lipid-based drug delivery systems can be used in the practice of the invention. For instance, multivesicular liposomes (MVL), multilamellar liposomes (also known as multilamellar vesicles or “MLV”), unilamellar liposomes, including small unilamellar liposomes (also known as unilamellar vesicles or “SUV”) and large unilamellar liposomes (also known as large unilamellar vesicles or “LUV”), can all be used so long as a sustained release rate of the encapsulated Aβ-interferer or p75 receptor-interferer can be established. In one embodiment, the lipid-based formulation can be a multivesicular liposome system. Methods of making controlled release multivesicular liposome drug delivery systems is described in PCT Application Ser. Nos. US96/11642, US94/12957 and US94/04490, the contents of which are incorporated herein by reference.

The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides. Preferably phospholipids including egg phosphatidylcholine, dipalmitoylphosphatidylcholine, distearoylphosphatidylcholine, dioleoylphosphatidylcholine, dipalmitoylphosphatidylglycerol, and dioleoylphosphatidylglycerol are used.

In preparing lipid-based vesicles containing an Aβ-interferer or p75 receptor-interferer, such variables as the efficiency of Aβ-interferer or p75 receptor-interferer encapsulation, lability of the Aβ-interferer or p75 receptor-interferer, homogeneity and size of the resulting population of vesicles, Aβ-interferer- or p75 receptor-interferer-to-lipid ratio, permeability, instability of the preparation, and pharmaceutical acceptability of the formulation should be considered (see Szoka, et al., Annual Reviews of Biophysics and Bioengineering, 9:467, 1980; Deamer, et al., in Liposomes, Marcel Dekker, New York, 1983, 27; and Hope, et al., Chem. Phys. Lipids, 40:89, 1986, the contents of which are incorporated herein by reference).

### Administration of the Pharmaceutically Acceptable Formulation

In one embodiment, the Aβ-interferer or p75 receptor-interferer is administered by introduction into the central nervous system of the subject, e.g., into the cerebrospinal fluid of the subject. In certain aspects of the invention, the Aβ-interferer or p75 receptor-interferer is introduced intrathecally, e.g., into a cerebral ventricle, the lumbar area, or the cisterna magna.

The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization, described in U.S. Pat. No. 436,742 the contents of which are incorporated herein by reference.

In another embodiment of the invention, the Aβ-interferer or p75 receptor-interferer formulation is administered into a subject intrathecally. As used herein, the term “intrathecal administration” is intended to include delivering an Aβ-interferer or p75 receptor-interferer formulation directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of an Aβ-interferer or p75 receptor-interferer to any of the above mentioned sites can be achieved by direct injection of the Aβ-interferer or p75 receptor-interferer formulation or by the use of infusion pumps. For injection, the Aβ-interferer or p75 receptor-interferer formulation of the invention can be formulated in liquid solutions, preferably in physiologically compatible buffers such as Hank's solution or Ringer's solution. In addition, the Aβ-interferer or p75 receptor-interferer formulation may be formulated in solid form and re-dissolved or suspended immediately prior to use. Lyophilized forms are also included. The injection can be, for example, in the form of a bolus injection or continuous infusion (e.g., using infusion pumps) of the Aβ-interferer or p75 receptor-interferer formulation.

### Duration and Levels of Administration

In another embodiment of the method of the invention, the pharmaceutically acceptable formulation provides sustained delivery, e.g., “slow release” of the Aβ-interferer or p75 receptor-interferer to a subject for at least one, two, three, or four weeks after the pharmaceutically acceptable formulation is administered to the subject.

As used herein, the term “sustained delivery” is intended to include continual delivery of an Aβ-interferer or p75 receptor-interferer in vivo over a period of time following administration, preferably at least several days, a week or several weeks. Sustained delivery of the Aβ-interferer or p75 receptor-interferer can be demonstrated by, for example, the continued therapeutic effect of the Aβ-interferer or p75 receptor-interferer over time (e.g., sustained delivery of the Aβ-interferer or p75 receptor interferer can be demonstrated by continued inhibition of neuronal cell death over time). Alternatively, sustained delivery of the Aβ-interferer or p75 receptor-interferer may be demonstrated by detecting the presence of the Aβ-interferer or p75 receptor-interferer in vivo over time.

In one embodiment, the pharmaceutically acceptable formulation provides sustained delivery of the Aβ-interferer or p75 receptor-interferer to a subject for less than 30 days after the Aβ-interferer or p75 receptor-interferer is administered to the subject. For example, the pharmaceutically acceptable formulation, e.g., “slow release” formulation, can provide sustained delivery of the Aβ-interferer or p75 receptor-interferer to a subject for one, two, three or four weeks after the Aβ-interferer or p75 receptor-interferer is administered to the subject. Alternatively, the pharmaceutically acceptable formulation may provide sustained delivery of the Aβ-interferer or p75 receptor-interferer to a subject for more than 30 days after the Aβ-interferer or p75 receptor-interferer is administered to the subject.

The pharmaceutical formulation, used in the method of the invention, contains a therapeutically effective amount of the Aβ-interferer or p75 receptor-interferer. A “therapeutically effective amount” refers to an amount effective, at dosages and for periods of time necessary, to achieve the desired result. A therapeutically effective amount of the Aβ-interferer or p75 receptor-interferer may vary according to factors such as the disease state, age, and weight of the subject, and the ability of the Aβ-interferer or p75 receptor-interferer (alone or in combination with one or more other agents) to elicit a desired response in the subject. Dosage regimens may be adjusted to provide the optimum therapeutic response. A therapeutically effective amount is also one in which any toxic or detrimental effects of the Aβ-interferer or p75 receptor-interferer are outweighed by the therapeutically beneficial effects. A non-limiting range for a therapeutically effective concentration of an Aβ-interferer or p75 receptor-interferer is 100 μM to 1 mM. It is to be further understood that for any particular subject, specific dosage regimens should be adjusted over time according to the individual need and the professional judgment of the person administering or supervising the administration of the Aβ-interferer or p75 receptor-interferer and that dosage ranges set forth herein are exemplary only and are not intended to limit the scope or practice of the claimed invention.

### In Vitro Treatment of Neuronal Cells

Neurons, e.g., CNS neurons, or isolated neuronal cells can further be contacted with a therapeutically effective amount of a Aβ-interferer or p75 receptor-interferer, in vitro. Accordingly, neuronal cells can be isolated from a subject and grown in vitro, using techniques well known in the art. Briefly, a neuronal cell culture can be obtained by allowing neuron cells to migrate out of fragments of neuronal tissue adhering to a suitable substrate (e.g., a culture dish) or by disaggregating the tissue, e.g., mechanically or enzymatically, to produce a suspension of neuronal cells. For example, the enzymes trypsin, collagenase, elastase, hyaluronidase, DNase, pronase, dispase, or various combinations thereof can be used. Trypsin and pronase give the most complete disaggregation but may damage the cells. Collagenase and dispase give a less complete disagregation but are less harmful. Methods for isolating tissue (e.g., neuronal tissue) and the disaggregation of tissue to obtain cells (e.g., neuronal cells) are described in Freshney R. I., Culture of Animal Cells, A Manual of Basic Technique, Third Edition, 1994, the contents of which are incorporated herein by reference.

Such cells can be subsequently contacted with an Aβ-interferer or p75 receptor-interferer at levels and for a duration of time as described above. Once inhibition of neuronal cell death has been achieved, these neuronal cells can be re-administered to the subject, e.g., by implantation.

States Characterized by Aβ-Induced and/or p75 Receptor-Mediated Neuronal Cell Death

The present invention further pertains to a method of treating a disease state characterized by Aβ-induced and/or P75 receptor-mediated neuronal cell death in a subject. As used herein, the term “state” is art recognized and includes a disorder, disease or condition characterized by Aβ-induced and/or p75 receptor-mediated neuronal cell death. Examples of such disorders include Alzheimer's Disease, dementias related to Alzheimer's disease (such as Pick's disease), Parkinson's and other Lewy diffuse body diseases, multiple sclerosis, amyotrophic lateral sclerosis, progressive supranuclear palsy, and spongiform encephalitis.

The invention is further illustrated by the following examples, which should not be construed as further limiting. The contents of all references, patents and published patent applications cited throughout this application are hereby incorporated by reference.

### EXAMPLES

NGF-differentiated PC-12 cells were treated with fibrillar Aβ₄₀ or fibrillar Aβ₄₂ in the presence or absence of Aβ-interferers. The percentage of dead cells were determined by MTT and SRB (rhodamine based dye—protein count) assays (as described in, for example, Rubinstein L. V. et al. (1990) J. Natl. Cancer Inst. 82 (13): 1113-8) after a 24 hour incubation. Cells were incubated with Aβ₄₀ with same weight compounds at 1:1 or 1:2—weight:weight ratio.

The contents of all references, issued patents, and published patent applications cited throughout this application, including the background, are hereby incorporated by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

What is claimed is:

1. A method for providing neuroprotection to a subject susceptible to or having a condition characterized by neurodegeneration, neuronal cell toxicity, neuronal cell death, decreased neuronal cell communication or senility comprising administering 3-amino-1-propanesulfonic acid or an acceptable salt thereof to the subject.

2. The method according to claim 1, comprising administering 3-amino-1-propanesulfonic acid.

3. The method according to claim 1, comprising administering 3-amino-1-propanesulfonic acid, sodium salt.

4. The method according to claim 1, wherein said 3-amino-1-propanesulfonic acid or acceptable salt thereof is formulated for oral administration.

5. The method of claim 1, wherein said condition is further characterized by the presence of neurotoxic Aβ peptides or proteins.

6. The method of claim 5, wherein the neurotoxic Aβ peptides or proteins is soluble Aβ.
